(19) United States
(12) Patent Application Publication
(10) Pub. No.: US 2011/0307035 A1
(43) Pub. Date: Dec. 15, 2011

(54) Phototherapy device

(75) Inventors: Yu-Chia Tsao, Taipei City (TW); Yi-Wen Yang, Taipei City (TW); Jung-Chien Chang, Sanxia Township (TW); Jia-Huey Tsao, Yingge Township (TW); Yen-Chun Chen, Hsinchu City (TW); Hsueh-Ching Shih, Xizhi City (TW)

(73) Assignee: Forward Electronics Co., Ltd., Taipei City (TW)

(21) Appl. No.: 12/926,147

(22) Filed: Oct. 28, 2010

(30) Foreign Application Priority Data
Jun. 9, 2010 (TW) 099118785

(51) Int. Cl. A61N 5/073

(52) U.S. Cl. 607/90; 607/88

(57) ABSTRACT

A phototherapy device is disclosed, which is driven by a power supply and includes: an LED module, driven by the power supply to emit therapeutic light; and a polarizer, disposed in a direction toward which the therapeutic light is emitted by the LED module. Accordingly, the phototherapy device according to the present invention can use light of low intensity to achieve therapeutic effect and thereby can be designed in a portable form.

### BACKGROUND OF THE INVENTION

1. Field of the Invention

The present invention relates to a phototherapy device and, more particularly, to a phototherapy device suitable for the application of light in low intensity.

2. Description of Related Art

With the improvement of the quality of the life, the cosmetology industry has developed quickly and phototherapy that can be used for treatment of acne, spot whitening, scar removal, wrinkle removal and whitening has become popular. A medicine journal reported that propionibacterium acnes, which cause redness and inflammation associated with acne, contain porphyrin, and free radicals can be generated by reaction between blue light (its wavelength ranges from about 400 nm to 470 nm) and porphyrin to eradicate propionibacterium acnes so as to reduce redness and inflammation associated with acne. In addition, red light (its wavelength ranges from about 600 nm to 700 nm) is helpful for wound healing and anti-inflammation; yellow light (its wavelength ranges from about 550 nm to 600 nm) can improve the circulation of skin cells and promote the regeneration of skin cells; and green light (its wavelength ranges from about 500 nm to 550 nm) can be used to regulate the function of skin glands and oil secretion and inhibit acne. Thereby, phototherapy can be performed by using light of a desired wavelength according to personal requirement to achieve a cosmetology or treatment object.

In addition to laser and pulsed light, ordinary light or LED light has been developed in phototherapy in place of the above-mentioned light of high intensity. However, the practical application of LED light has some problems. For example, the intensity of LED light is low and LED light sources of low performance cannot achieve therapy. On the other hand, LED light sources of high performance are disadvantageous to the development of portable phototherapy systems with reduced volume and weight and thereby cannot be used in place of pulsed light.

Thereby, it is desirable to develop an LED phototherapy device of significantly reduced volume and weight, which is suitable for home use.

### SUMMARY OF THE INVENTION

The object of the present invention is to provide a portable phototherapy device in which a light source of low intensity is used to achieve phototherapy effect.

To achieve the object, the present invention provides a phototherapy device driven by a power supply, including: an LED module, driven by the power supply to emit therapeutic light; and a polarizer, disposed in a direction toward which the therapeutic light is emitted by the LED module.

Accordingly, the present invention uses a polarizer to enhance transmittance of therapeutic light, such that therapeutic light can be transmitted to the depths of illuminated sites to achieve phototherapy effect even using light of low intensity. Thereby, the phototherapy device according to the present invention can use a light source of low intensity to significantly reduce its volume and weight and thus can be self-applied by users.

The phototherapy device according to the present invention may further include: a housing having a light outlet, where the polarizer is disposed at the light outlet of the housing and the LED module is disposed in an interior of the housing.

The phototherapy device according to the present invention may connect to an outer power supply or use a battery as a power supply to drive the LED module. Herein, the battery may be a rechargeable battery, an ordinary battery or a micro battery. Preferably, the phototherapy device according to the present invention uses a battery as a power supply and thereby is advantageous to a portable design. Accordingly, in the present invention, the housing of the phototherapy device may have a power supply receiving part to receive the power supply.

In the present invention, the LED module may include at least one LED component and a circuit board, where the LED component electrically connects to the circuit board and the circuit board electrically connects to the power supply to drive the LED component. Herein, the phototherapy device according to the present invention may further include a control module, which electrically connects to the circuit board to switch the LED component into a bright or dark state.

In the present invention, the LED module may include a plurality of LED components capable of emitting therapeutic lights with various wavelengths. For example, the LED components may emit therapeutic lights of 400-440 nm, 440-470 nm, 500-550 nm, 550-600 nm, 600-700 nm and 700-1000 nm, respectively. Accordingly, users can switch the LED components respectively into a bright or dark state by operating the control module to make the LED module emit therapeutic light with a desired wavelength according to personal requirement.

In the present invention, the polarizer may be a linear polarizer, such that therapeutic light emitted by the LED module can be transformed into linearly polarized light.

In the present invention, the intensity of therapeutic light emitted by the LED module may range from about 2 mW/cm² to about 4 mW/cm² to be advantageous to a portable design.

### BRIEF DESCRIPTION OF THE DRAWINGS

FIG. 1 shows a system diagram of a phototherapy device according to a preferred example of the present invention;

FIG. 2 shows a schematic diagram of a phototherapy device according to a preferred example of the present invention;

FIG. 3 shows a schematic diagram of a phototherapy device according to another preferred example of the present invention;

FIG. 4 shows a schematic diagram of a phototherapy device according to yet another preferred example of the present invention; and

FIG. 5 shows a schematic diagram of propionibacterium acnes being cultured in a dish.

### DETAILED DESCRIPTION OF THE PREFERRED EMBODIMENT

Hereafter, examples will be provided to illustrate the embodiments of the present invention. Other advantages and effects of the invention will become more apparent from the disclosure of the present invention. It should be noted that these accompanying figures are simplified. The quantity, shape and size of components shown in the figures may be modified according to practically conditions, and the arrangement of components may be more complex. Other various aspects also may be practiced or applied in the invention, and various modifications and variations can be made without departing from the spirit of the invention based on various concepts and applications.

### Example 1

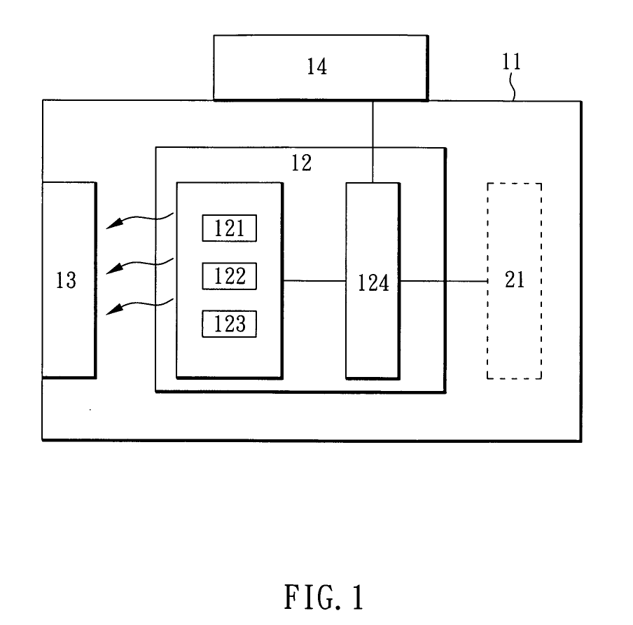

With reference to FIG. 1, there is shown a system diagram of a phototherapy device according to the present example. As shown in FIG. 1, the phototherapy device of the present example is driven by a power supply 21 and includes: a housing 11; an LED module 12, driven by the power supply 21 to emit therapeutic light and disposed in an interior of the housing 11; a polarizer 13, disposed in a direction toward which the therapeutic light is emitted by the LED module 12; and a control module 14, electrically connecting to the LED module 12 to switch the LED module 12 into a bright or dark state. In detail, the LED module 12 includes a plurality of LED components 121, 122 and 123 and a circuit board 124. Herein, the LED components 121, 122 and 123 electrically connect to the circuit board 124; the circuit board 124 electrically connects to the power supply 21 to drive the LED components 121, 122 and 123; and the control module 14 electrically connects to the circuit board 124 to switch the LED components 121, 122 and 123 into a bright or dark state. Additionally, the polarizer 13 used in the present example is a linear polarizer.

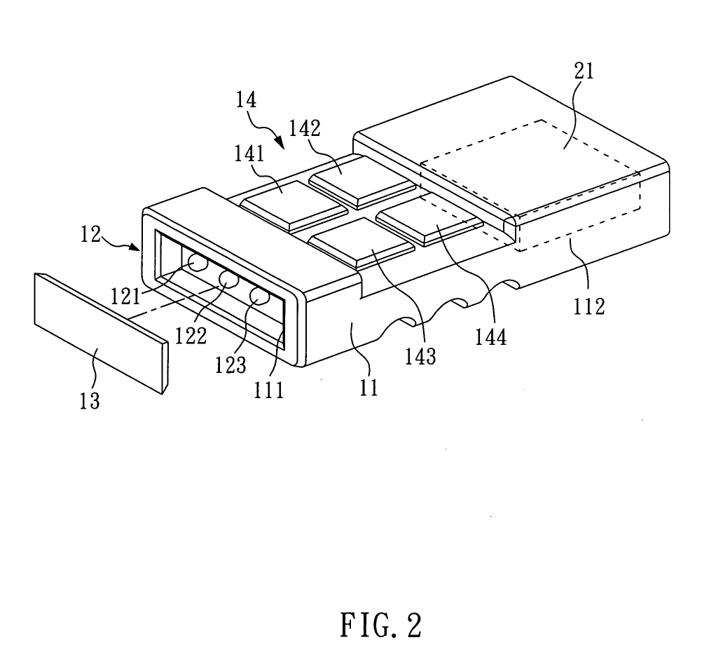

With reference to FIG. 2, there is shown a schematic diagram of a phototherapy device according to the present example. As shown in FIG. 2, the housing 11 of the phototherapy device according to the present example has a light outlet 111. Herein, the polarizer 13 is disposed at the light outlet 111 of the housing 11, and the LED module 12 is disposed in an interior of the housing 11. In addition, the phototherapy device of the present example uses a battery as a power supply 21. As shown in FIG. 2, the housing 11 of the phototherapy device further has a power supply receiving part 112 to receive the power supply 21.

As shown in FIG. 2, according to the present example, the LED module 12 includes a plurality of LED components 121, 122 and 123, which emit therapeutic lights of 440 nm-470 nm, 500 nm-550 nm and 600 nm-700 nm in low intensity (about 2 mW/cm²), respectively, and the control module 14 includes a plurality of switch components 141, 142 and 143. Accordingly, users can switch the LED components 121, 122 and 123 respectively into a bright or dark state via the switch components 141, 142 and 143 of the control module 14 to make the LED module 12 emit therapeutic light with a desired wavelength according to personal requirement.

For example, if users want to treat acne through therapeutic light of 440 nm to 470 nm, they can press the switch component 141 corresponding to the LED component 121 to allow the LED component 121 to emit therapeutic light of 440 nm to 470 nm. If users want to use therapeutic light of 500 nm to 550 nm to reduce darkness of skin, they can press the switch component 144 to switch the pressed switch component 141 into a non-pressed state and thereby control the LED component 121 not to emit therapeutic light, and then press the switch component 142 corresponding to the LED component 122 to allow the LED component 122 to emit therapeutic light of 500 nm to 550 nm. Similarly, in the case of using therapeutic light of 600 nm to 700 nm to promote wound healing, users can first press the switch component 144 to control the LED component 122 not to emit therapeutic light, and then press the switch component 143 corresponding to the LED component 123 to allow the LED component 123 to emit therapeutic light of 600 nm to 700 nm. Also, users can press two or more switch components simultaneously to allow the LED module 12 to emit therapeutic lights of two or more wavelengths. Finally, these pressed switch components can be switched into being non-pressed by pressing the switch component 144 to make the LED module 12 not emit any therapeutic light.

### Example 2

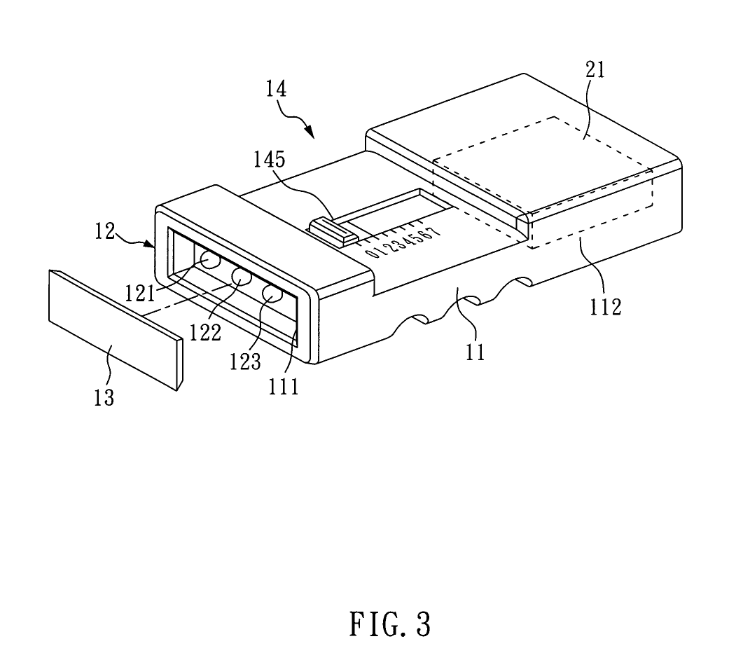

Please refer to FIG. 3. The phototherapy device according to the present example is the same as that illustrated in Example 1, except that the control module 14 according to the present example is designed to have a sliding switch component 145. Specifically, the LED module 12 does not emit therapeutic light in the case of sliding the switch component 145 to the location “0”; the LED component 121 emits therapeutic light of 440 nm to 470 nm in the case of sliding switch component 145 to the location “1”; the LED component 122 emits therapeutic light of 500 nm to 550 nm in the case of sliding switch component 145 to the location “2”; the LED component 123 emits therapeutic light of 600 nm to 700 nm in the case of sliding switch component 145 to the location “3”; the LED components 121 and 122 simultaneously emit therapeutic lights of 440-470 nm and 500-550 nm in the case of sliding switch component 145 to the location “4”; the LED components 121 and 123 simultaneously emit therapeutic lights of 440-470 nm and 600-700 nm in the case of sliding switch component 145 to the location “5”; the LED components 122 and 123 simultaneously emit therapeutic lights of 500-550 nm and 600-700 nm in the case of sliding switch component 145 to the location “6”; and the LED components 121, 122 and 123 simultaneously emit therapeutic lights of 440-470 nm, 500-550 nm and 600-700 nm in the case of sliding switch component 145 to the location “7”.

### Example 3

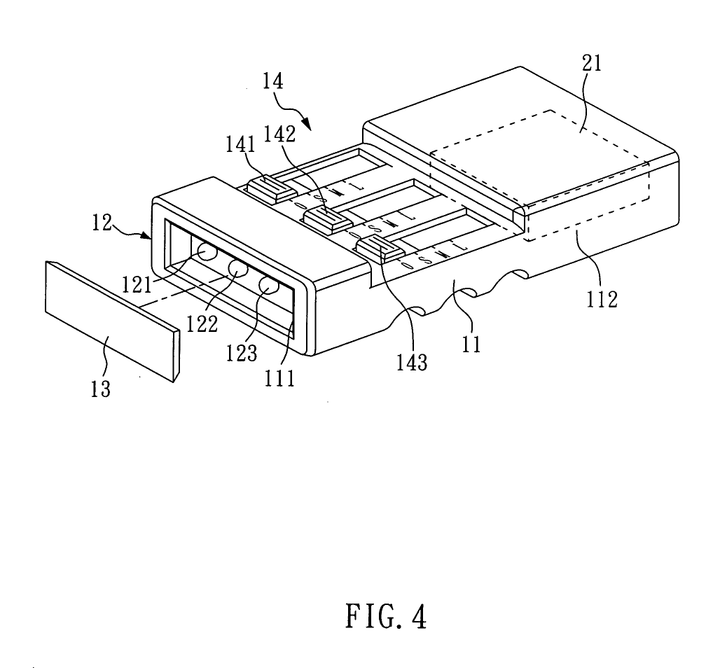

Please refer to FIG. 4. The phototherapy device according to the present example is the same as that illustrated in Example 1, except that the control module 14 according to the present example is designed to have a plurality of sliding switch components 141, 142 and 143. Herein, the switch components 141, 142 and 143 according to the present example can be operated not only to switch the LED components 121, 122 and 123 into a bright or dark state, but also to modulate the intensity of therapeutic light. As shown in FIG. 4, the LED components 121, 122 and 123 correspond to the switch components 141, 142 and 143, respectively, and the switch components 141, 142 and 143 can be respectively operated to switch the LED components 121, 122 and 123 into a bright or dark state and to moderate light intensity. Taking the switch component 141 as an example, the LED component 121 emits no therapeutic light when the switch component 141 is slid to the location “0”; the LED component 121 emits therapeutic light of 440 nm to 470 nm in an intensity of 2 mW/cm² when the switch component 141 is slid to the location “S”; the LED component 121 emits therapeutic light of 440 nm to 470 nm in an intensity of 3 mW/cm² when the switch component 141 is slid to the location “M”; and the LED component 121 emits therapeutic light of 440 nm to 470 nm in an intensity of 4 mW/cm² when the switch component 141 is slid to the location “L”. Similarly, the LED components 122 and 123 can be respectively operated by the switch components 142 and 143 to be dark or to emit therapeutic lights of 500-550 nm and 600-700 nm in various intensities.

### Test Example

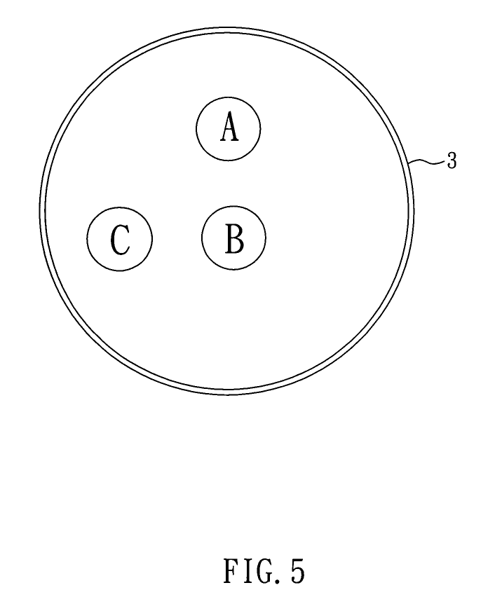

The results for inhibiting propionibacterium acnes were observed by illuminating propionibacterium acnes with blue LED light (400-420 nm and 460 nm) in various intensities. First, propionibacterium acnes were cultured at the regions A, B and C of the dish 3 in the absence of oxygen, as shown in FIG. 5. Then, the region B of the dish 3 was illuminated by the light source in a short distant at 37° C., and the propionibacterium acnes at the region B of the dish 3 were observed to evaluate the effect of the light source for inhibiting acnes in comparison with a control group in which no illumination was performed. If the result shows that there are no acnes being generated after illumination for 24 hours, it confirms that the light has the effect for inhibiting acnes. On the other hand, f the result shows that there is acnes being generated after illumination for 24 hours, it confirms that the light has no effect for inhibiting acnes. The test example 1 used a light source in high intensity (about 6.5 mW/cm²) to illuminate acnes; the test example 2 used a light source in middle intensity (about 4.0 mW/cm²) to illuminate acnes; the test example 3 used a light source in low intensity (about 2.0 mW/cm²) to illuminate acnes; and the test example 4 used a light source in low intensity (about 2.0 mW/cm²) and a polarizer to illuminate acnes. The results are shown in Table 1. Herein, ⊚ refers to that the light has the effect for inhibiting acnes; and × refers to that the light has no effect for inhibiting acnes.

TABLE 1

| Test Example | 400-420 nm | 460 nm |
|---|---|---|
| 1 | ⊚ | ⊚ |
| 2 | ⊚ | ⊚ |
| 3 | X | X |
| 4 | ⊚ | ⊚ |

From Table 1, it can be known that the polarizer can enhance the transmittance of light and achieve therapeutic effect even using light of low intensity.

The above examples are intended for illustrating the embodiments of the subject invention and the technical features thereof, but not for restricting the scope of protection of the subject invention. The scope of the subject invention is based on the claims as appended.

What is claimed is:

1. A phototherapy device driven by a power supply, comprising:
an LED module, driven by the power supply to emit therapeutic light; and
a polarizer, disposed in a direction toward which the therapeutic light is emitted by the LED module.

2. The phototherapy device as claimed in claim 1, wherein the LED module comprises at least one LED component and a circuit board, therewith the at least one LED component electrically connecting to the circuit board and the circuit board electrically connecting to the power supply to drive the at least one LED component.

3. The phototherapy device as claimed in claim 2, further comprising: a control module, electrically connecting to the circuit board to switch the at least one LED component into a bright or dark state.

4. The phototherapy device as claimed in claim 3, wherein the LED module comprises a plurality of LED components capable of emitting therapeutic lights with various wavelengths.

5. The phototherapy device as claimed in claim 4, wherein the LED components are respectively switched into a bright or dark state via the control module.

6. The phototherapy device as claimed in claim 1, further comprising: a housing having a light outlet, wherein the polarizer is disposed at the light outlet of the housing and the LED module is disposed in an interior of the housing.

7. The phototherapy device as claimed in claim 6, wherein the housing further has a power supply receiving part to receive the power supply.

8. The phototherapy device as claimed in claim 1, wherein the polarizer is a linear polarizer.

9. The phototherapy device as claimed in claim 1, wherein the therapeutic light emitted by the LED module ranges from 2 mW/cm² to 4 mW/cm² in intensity.

10. The phototherapy device as claimed in claim 1, wherein the therapeutic light emitted by the LED module ranges from 400 nm to 440 nm, 440 nm to 470 nm, 500 nm to 550 nm, 550 nm to 600 nm, 600 nm to 700 nm, 700 nm to 1000 nm or a mixture thereof in wavelength.
